(19) United States
(12) Patent Application Publication
(10) Pub. No.: US 2006/0281977 A1
(43) Pub. Date: Dec. 14, 2006

(54) Diagnostic and treatment planning calculator

(76) Inventor: Michael Soppet, Dothan, AL (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/442,091

(22) Filed: May 26, 2006

Related U.S. Application Data
(60) Provisional application No. 60/688,777, filed on Jun. 9, 2005.

(51) Int. Cl. A61B 5/00; G06Q 10/00

(52) U.S. Cl. 600/300; 705/2

(57) ABSTRACT

Methods and apparatus are provided to help medical personnel receives and input a first interface that receives patient-specific information, a second interface that displays a plurality of treatment options, a processor that executes software that projects clinical outcomes for at least first and second ones of the treatment options as a function of historic data and the patient-specific information. and a third interface that displays at least the first and second ones of the treatment options.

This application claims priority to U.S. provisional application Ser. No. 60/688,777 filed Jun. 9, 2005.

### FIELD OF THE INVENTION

The field of the invention is medical diagnosis and treatment.

### BACKGROUND

With all the available treatment options in medicine today, physicians and patients face a plethora of choices in determining the best treatment regime given a particular diagnosis. This often leads both parties to make hasty and non-collaborative decisions, which can cause patient confusion and noncompliance. Under severe time pressure to process large patient flows, physicians face limitations in determining diagnostic and treatment options in a concise and accurate way. For their part, patients would prefer to have greater knowledge about the diagnostic or treatment options, and to have a greater degree of collaboration in the decision making process. Among other things, many patients have a great desire to weigh for themselves the risks of those various options, and their individual likelihood of success with each diagnostic attempt or treatment proposed.

A threshold problem is the amount of time a patient has with a physician. Physicians are spending less face time with patients for diagnosis and even less time in assessing the different treatment options that are presented. The average length of face time with a physician in a medical office is only eleven minutes. Depending on the diagnosis and the available treatment options, physicians might not be able to explain all the clinical outcomes to the patients in such a short amount of time, which can leave the patient confused and unsatisfied.

Even with more face time, it is often difficult for physicians to provide a broad comparison of the different treatment options as these correlates to the patient specifically. Ideally, the physician's recommendations should be evidence based. However, current practice makes it difficult for an individual physician to easily locate and display this information for the individual patient in real time at the point of care. Physicians may well have considerable knowledge regarding diagnostic and treatment options, and even have an ability to communicate this knowledge on an aggregate basis. But they can still lack specific tools to individualize the particular knowledge to a specific patient with any degree of certainty. When the physician offers a best guess estimate of the effectiveness of a diagnostic or treatment plan in hand, patients often are confused as to the benefits and costs of a given plan, including severity and frequency of symptoms, laboratory testing, side effects, and monetary costs. This can all lead to low compliance on the part of the patients.

As shown in U.S. Pat. No. 5,724,580 to Levin et al. (1998), U.S. Pat. No. 6,409,664 to Kattan et al: (2002), and U.S. 2004/0248151 to Bacus et al. (2004), computer programs have been designed to select a preferred treatment option based on patient specific information. However, none of the prior art software proposes alternative treatment options or offers comparisons between them. Thus, there still is no readily accessible means for a patient to compare the various treatment options for their particular circumstance. Other programs, such as those described in U.S. Pat. No. 5,860,917 to Comanor et al. (1999) and U.S. Pat. No. 6,56,114 to Poulsen et al. (2003), attempt to display alternative treatment choices and draw conclusions as to treatment options. But those programs only allow a user to select a specific choice. They do not show the expected outcomes from each of a variety of alternatives

Another problem limiting a physician's delivery of care is that the physician-patient relationship has traditionally been asymmetric. Physicians have the benefit of knowledge outside the purview of patients. As a result, the traditional diagnostic and treatment paradigm has been wholly designed by the physician and is thus one-sided and non-collaborative. Programs such as the one in the '917 patent are designed only for the benefit of the physician to analyze statistical models of a treatment plan, leaving the patients to feel disconnected from the treatment strategy selected, and often resistant to completing the treatment plan. Studies show that up to 30% of prescriptions written by physicians are never even filled by the patient. Moreover, patients often have lingering questions on a treatment plan, and second thoughts after speaking with friends, family members, reading lay information on the subject, or searching the Internet. Full disclosure and discussion of all aspects of the proposed plan with the physician, when the treatment plan is being decided upon, allow the patient to claim co-authorship of the plan. Authorship equates to ownership and with that, the patient is more likely to follow through with the treatment plan.

Another problem with the prior art relates to the lack of portability and size of the program. While some doctor's office are equipped with desktop or laptop computers, most medical facilities for patient visits do not have access to a computer. Many of the programs designed to help physicians select different treatment options must be used on a desktop or laptop computer. Therefore, a device that is easy to use, with a high speed of processing evidence based data is ideal for medical personnel to assess diagnosis and treatment options at the point of care.

None of the known methods, apparatus, devices and systems have been particularly effective in solving these problems. Thus, there is still a need for an improved apparatus and methods to communicate patient-specific information, diagnosis, correlate treatment options and expected clinical outcomes in a convenient fashion.

This and all other referenced extrinsic materials are incorporated herein by reference in their entirety. Where a definition or use of a term in an incorporated reference is inconsistent or contrary to the definition of that term provided herein, the definition of that term provided herein applies and the definition of that term in the reference does not apply.

### SUMMARY OF THE INVENTION

The present invention provides apparatus, systems and methods in which an electronic device provides lists of treatment options and corresponding projected clinical outcomes for specific circumstances of individual patients.

Preferred embodiments have a first interface that receives patient-specific information, a second interface that displays a plurality of treatment options, a processor that executes software that projects clinical outcomes for at least first and second ones of the treatment options as a function of historic data and the patient-specific information, and a third interface that displays at least the first and second ones of the treatment options.

In especially preferred embodiments, one or more of the interfaces comprises an electronic display, and the third interface comprises a display or printout. One or more of the interfaces is also preferably presented in spreadsheet format. All possible symptoms are contemplated to be used with the present inventive subject matter, including for example a change in severity, frequency, or duration of a symptom, or a change in a laboratory value or other sign. Preferred embodiments can also project side effects, monetary or other costs. Thus, medical professionals can use the comparison as a basis from which to discuss at least some of the treatment options and at least some of the clinical outcomes with the patient.

In yet another preferred embodiment, a fourth interface receives a diagnosis and a software projects the clinical outcomes based from the diagnosis. The fourth interface also lists all the diagnostic possibilities by correlating with the first interface of patient-specific information. The fourth interface also correlates costs with the diagnostic possibilities by correlating the first second ones of the treatment options. Preferably, the treatment options includes administration of a drug, lifestyle change or a combination thereof.

With respect to the device, it is generally preferred to be a pocket-size housing that couples to a display screen, control buttons, microprocessor, and a memory. Furthermore, the device has sufficient processing power and memory such that the device does not need to communicate with an external source to produce the comparison. Alternatively, the device has the ability to communicate to an external source to produce the comparison on clinical outcomes.

### BRIEF DESCRIPTION OF THE DRAWING

FIG. 1 is a flowchart according to the inventive subject matter.

FIG. 2 is a device according to the inventive subject matter.

FIG. 3 is an illustration of implementing the inventive subject matter.

### DETAILED DESCRIPTION

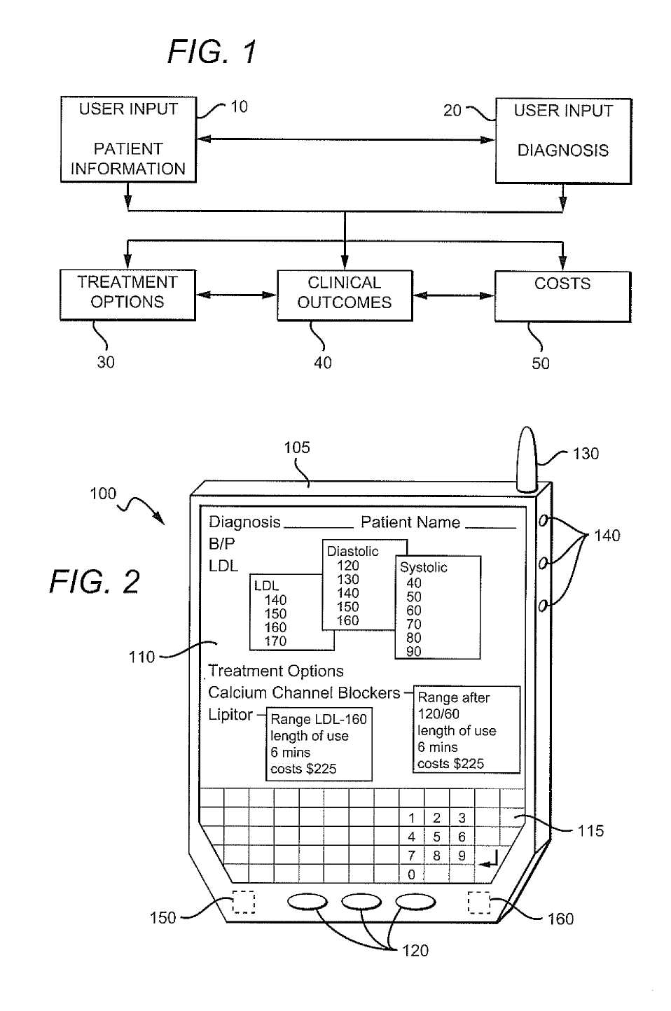

In FIG. 1, a flowchart generally includes steps for capturing patient-specific information 10, diagnosis 20, treatment options 30, clinical outcomes 40, and costs 50.

Patient-specific information 10 is any information specific to the patient that could affect any of the diagnosis, treatment options, clinical outcomes, or costs. Typically the patient is a human, but it will be appreciated that the patient can be any mammal such as, dogs, cats, cows, sheep, horses, pigs and the like. The patient-specific information could include, for example, demographic information such as age, gender, race or ethnicity (or breed in the case of animals). It could also include signs and symptoms, past medical history, and so forth. Thus, for a patient suffering from high blood pressure, the patient-specific information would likely include blood pressure measurements and so forth. Patient-specific information could even include insurance information because such data could affect the costs.

The patient-specific information would presumably be entered by a doctor, nurse, or other health-care professional, but could also be entered by a patient, family member, or anyone else. Additionally or alternatively, any or all of such information could be received from a medical records server, or other electronic source remotely. Since the number of different signs, symptoms and other patient-specific information fields is very large, the system will almost certainly present only a small subset of field names at any given time. Data entry fields associated with the field names can be one or any combination of open-ended text or number fields, drop-down choices, and so forth.

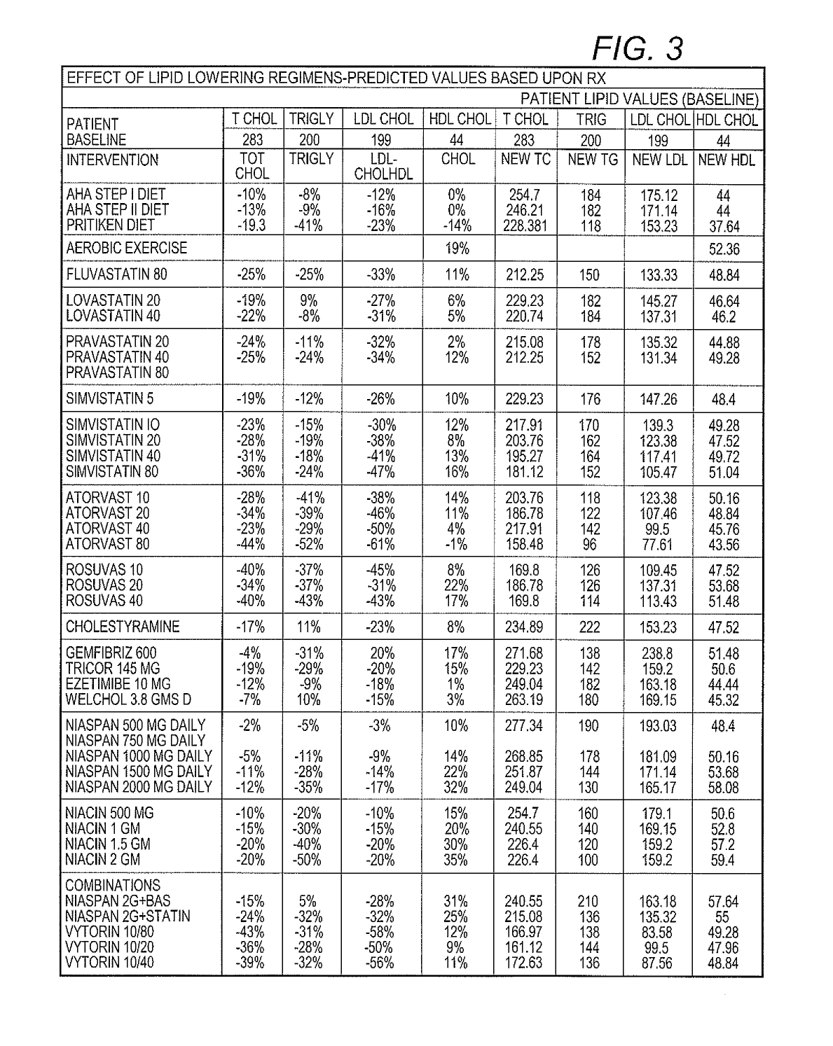

Diagnosis 20 should be interpreted herein to include any sort of conclusion as to a disease condition of the patient, whether expressed formally or informally, whether tentative or established, and so forth. For example, diagnosis might include any diseases, such as high-blood pressure, hyperlipidemia, hypertension, osteoporosis, hypercholesterolemia. The patient-specific information of step 10, and the diagnostic data of step 20 are entered into the system by the health care professional via a device as described in FIG. 2 and displayed in FIG. 3. Alternatively, diagnosis 20 can be derived and processed based on the patient-specific information entered. Preferably, a user entering a laboratory value specific to a disease will trigger the corresponding diagnosis. For example, if a user has selected blood pressure readings, then a selection of diagnosis are presented, such as hypertension.

Diagnosis 20 and patient-specific information can also work in reverse. In preferred embodiments the selection of a diagnosis triggers a change in the field names used to capture patient-specific information. Thus, in the case of a diagnosis of hypertension, the field names would likely include blood-pressure readings, but in the case of osteoporosis the field names would likely include bone density readings.

Treatment options 30 are a list of treatments that can be efficacious in remediating aberrant patient-specific information, and/or in treating the indicated disease. Thus, treatment options 30 can include drug treatment regimens, surgery physical therapy, counseling, lifestyle changes, and even non-traditional treatments such as acupuncture, chiropractic, and the like. In appropriate cases such as in hyperlipoproteinemia, contemplated systems and methods can also provide greater granularity as to a class of treatments, such as providing separate entries for a high dosage of HMA-CoA Reductase Inhibitor (so called statin) drugs and a low dosage of the same statin, length of treatment time, and number of office visits.

Clinical outcomes 40 is a list of projected changes in the clinical picture. Outcomes could be presented in any suitable fashion, including for example a change in a laboratory value, or a change in frequency, duration, or severity of a sign or symptom. The general idea is to provide patients (and health care professionals) with a comparative view of how useful the various treatment options are likely to be. Thus, if a patient is presented with a choice of: (a) reducing cholesterol by 30 points by adopting a level 2 American Heart Association diet for a year, and (b) reducing cholesterol by 80 points by taking—mg of—for two months, the patient might chose either one, or even both of those options. But a significant feature is that the patient (or owner in the case of animals) is provided with a convenient summary with which to make an intelligent decision. The physician and patient (or owner) can cooperatively assess various treatment strategies based upon the individual expected results within specified confidence intervals at the point of care.

Another aspect of clinical outcomes 40 can be side effects. It is contemplated that patients will be much more compliant with respect to their treatment regimens if they have taken an active part in selecting the treatment, and in weighing for themselves the relative benefits and costs of those regimens. Providing patients with likely side effects of the various treatments allow the patient to review all the clinical outcomes at once. Alternatively, the outcome data is displayed on a device as described in FIG. 2 and FIG. 3 and can be further transmitted to an external network or device.

Costs 50 is really another type of outcome, but it is logically distinct from clinical outcomes 40 because the effect is monetary rather than clinical. Costs 50 is helpful for the patient and the health care professional in determining the benefits and the costs of the different treatment options.

Still another type of costs is side effects. It is contemplated that a user (doctor, patient, owner, administrator, etc) can be presented with lists of likely side effects of any of the treatment options, which for lack of space on the interface may well be presented on a linked or other second page. In especially preferred embodiments a user could also be presented some measure of the probabilities of occurrence of the various side effects. It is also possible to combine cost information with side effects information. This can be accomplished by accessing the patient's insurance plan formulary to determine the total out-of-pocket costs for each therapeutic intervention, along with the suggested follow up laboratory studies, their costs, and the cost of physician follow-up visits for a year or other period. Total insurance company costs can similarly be calculated and displayed or transmitted to the company. Thus, a cost estimate can be generated for the treatment option which the patient and physician jointly agree upon, a prescription can be printed or electronically generated, the insurance company can be notified of the intended treatment, and give authorization and the patient may proceed to the pharmacy for initiation of treatment. Still further, the total number of physician visits, laboratory tests, and dollars spent in achieving the target result, and unfilled prescriptions or partially administered prescriptions can be assessed and planned.

In short, systems, devices and methods contemplated herein can provide a “one stop shopping” opportunity for both patient and physician in selecting a treatment option. Patient and physician satisfaction will also increase through this method. Alternatively, the insurance company may notify its pharmacy benefits manager to dispense the medication immediately by mail to the patient's home.

In an exemplary configuration as depicted in FIG. 2, a device 100 generally comprises a housing 105 with a display screen 110, a plurality of control buttons 120, an antenna 130, various connector ports 140, a processor 150, and a memory 160.

Housing 105 should be interpreted generically as representing any enclosure that contains parts and gives structure. In this case the housing is sufficiently large to provide adequate interfaces for both input and output. Nevertheless, housing 105 is preferably portable, which comprehends everything from pocket-size to tablet size. Portability is contemplated to be especially useful in hospital and medical office environments because patients are typically located throughout the facility, and not necessarily in locations near a desk top or other non-portable computers or terminals. Thus, it is contemplated that medical personnel could carry around the inventive devices in a jacket, briefcase, purse, pants, or even a shirt pocket.

It is also contemplated that housing 105 can be made of any material that is sufficiently durable. Here again this feature can be extremely important in a hospital or medical office environment because the devices can be expected to encounter numerous insults, from dropping, to being bumped, and so forth. It may even be useful for the housing to provide some measure of water resistance, or even water proofing.

Display screen 110 is a surface on which text or picture is projected for viewing. Such displays can have two basic functions; (1) a display function; and (2) a data entry function. It is contemplated that the display function depicts texts, images, and other graphic representation of data received. Text information can be displayed on the screen in a data entry fields associated with the field names or any combination of open-ended text or number fields, drop-down menu, and so forth. Images can be used to demonstrate different functions selected by a user, such as an image of a person can indicate patient-specific information. Furthermore, a graphic representation can be useful in displaying a clinical outcome. Since time is always a factor in treating patients and the more data is shown on a screen, the more efficiently the doctor can be in assessing the data. Preferably, all the pertinent information is displayed on the screen at once.

As to the data entry function, the display screen allows for multiple data entry inputs, such as using a touch sensitive interface, inputting via a drop down menu and entering text via a hard keyboard. Like many portable devices, the touch sensitive function (using a pen or finger) can have a soft alphabetic keyboard which allows different screens to display different keyboard functions. For example, when inputting patient-specific information, the display screen might have soft keyboards for demographic fields such as age, weight, height and so forth, and when a user selects the treatment options, the display screen might have soft keyboards for fields such as drug treatment, lifestyle change, and so forth. The user can then select accordingly. Also, the touch sensitive function can recognize not only the touch of the screen but also the handwriting of the user. Physicians have been accustomed to using their own handwriting in prescribing medications and treatment options. This will allow them to have familiarity with the device.

Drop-down menus can be used to provide choices for selected fields. For example, when a physician needs to enter a patient's blood pressure level, a drop-down menu of the display screen could advantageously show systolic pressure ranges from 250-70 mm Hg, and diastolic pressures of 40-120 mm Hg, and so forth. The physician then can select the range accordingly. The drop-down menus can be color highlighted, or can be adapted in any other manner to assist in presenting the data. Specific functions of the display screen can be augmented or replaced by hard keys.

Devices are preferably designed from an ergonomic perspective. For example, factors such as color versus black white, size and pixel density, hand-shape, variously colored housings and so forth can all add enjoyment to the use of the device. In an especially preferred embodiment, the display screen has a color screen and high pixel density (using liquid crystal or plasma) to project a more clear and vibrant display for viewing. However, it should be recognized that black and white and other pixel densities are also deemed suitable.

The size and the shape of the display should at least be large enough for convenient manual usage. This includes everything from a PDA-sized device to a tablet sized laptop. Devices with different colored housings can make it simple for multiple doctors practicing in the same setting to distinguish one device from another. This can also serve to protect patient confidentiality.

Control buttons 120 allow a user to input data information and manipulate data to generate outcomes. Particularly preferred devices include control buttons to transmit, execute, store, display, print and so forth. Control buttons can be located anywhere on the device. Control buttons can be built in or used in lieu of a display screen 110 with touch-pad capabilities. In preferred aspects, control buttons further include: an on/off switch, an execute button, or a clear button. Control buttons can be of a different color than the housing and preferably be on the front of the device.

Whether embodied in control buttons or in some other manner, it is contemplated that devices can include some sort of security control requiring a password, and some sort of automatic turnoff feature that activates after a certain amount of inactivity.

Antenna 130 transmits and receives data to an external source, which for example, can be a medical records server Thus, instead of inputting patient-specific information, a doctor can simply pull the previously stored patient's information. Similarly, the antenna can transmit clinical outcome data back to the medical history record server for record keeping. Physicians can presumably also send data to each other. As is well known in the art, antennas can be built into the treatment device or exposed as shown in FIG. 2. The size and shape of antennas are determined primarily by the frequency of the signal they are designed to receive.

Connector ports 140 can also be employed to communicate with a suitable external device, whether by hardwire or wirelessly. Particularly preferred wireless interfaces include a radio transceiver (e.g., interface following the ‘blue tooth’ standard) or an opto-electronic transceiver (e.g., an IR-transceiver).

In a preferred embodiment, the device has sufficient processing power and memory such that the device does not need to communicate with an external source to produce the comparison. However, connector ports 140 can be employed to communicate with an alternative interface that allow communications directly to an external source. An alternative interface that is preferably a standard electronic plug-type is commonly a RS232. However, various alternative interfaces are also contemplated and include an USB interface, an IDB-C interface, an ISO11898 compliant CAN interface, or an IEEE1394 interface. With respect to the transferring any data between the device and an external source, it is generally preferred that the external source will adopt the data transfer protocol provided by the device for the respective connector ports. Thus, preferred data transfer protocols will especially include RS232-compatible data transfer protocols. With respect to the external source, it should be appreciated that numerous external sources are suitable for use herein, and appropriate sources may include patient medical history server, personal desktops and laptops, and other handheld devices, such as a personal device assistants (PDA). With respect to PDAs, a variety of PDAs can receive and transfer data using PDAs with a Windows CE operating system, a Palm operating system, or any other operating system that is appropriate for use in a hand held device.

Microprocessor 150 is any component of a computer system which manipulates data. The processor does: receives the data and sort it out, display the data, and process the data to match what the user is asking it to do. Microprocessors are advantageously included in at least the housing of the device to facilitate the communications of all data. The microprocessor may operate a RAM, ROM, or other data storage device.

In a preferred embodiment of the present invention the microprocessor is programmed to determine the diagnosis upon receiving the patient-specific information. Similarly, the microprocessor is programmed to determine treatment options upon receiving either the patient-specific information or the diagnosis data. Additionally, the microprocessor is programmed to determine the preferred clinical outcomes from the patient-specific information, the diagnosis, and the treatment options and other data inputted and/or received by the device. The microprocessor advantageously derives a mathematical relationship between the patient specific information, the diagnosis and the treatment options to obtain an ideal clinical outcome.

The data received by the microprocessor can be transmitted over the network and preferably transmitted via the Internet but may also be transmitted by telephone line, radio, pager, two-way pager, cable, and any other suitable communication mechanism.

It is generally contemplated that the microprocessor and the memory may vary considerably and that a particular configuration of such elements will predominantly depend on the type of computations employed.

Similarly, contemplated memory 160 will vary depending on the particular configuration of the device, and particularly suitable memory include commercially available micro hard drives with a 1-inch disc (which may preferably have a capacity of more than 1 GB), flash memory cards with a capacity of up to 128 MB and more, and other transient and/or permanent memory units. Likewise, suitable memory may include SDRAM, SIMM, DIMM, etc. with a capacity of at least 32 MB, more preferably at least 64 MB, and most preferably above 128 MB.

Furthermore, depending on the particular configuration and size of the device, it is contemplated that the device is configured to have a power source. The power source may vary considerably. For example, where the device is relatively large, suitable power sources may include a rechargeable battery. On the other hand, where the device is relatively small appropriate power sources especially include an external power source (e.g., transformer for wall outlet or cigarette lighter adapter).

### EXAMPLES

The following examples illustrate particularly embodiments of the present inventive subject matter, and aid those of skill in the art in understanding and practicing the inventive subject matter. They are set forth for explanatory purposes only, and are not to be taken as limiting the present inventive subject matter in any manner.

### Example 1

### Patient with Hyperlipoproteinemia and Risk of Cardiovascular Disease

One embodiment of the present inventive subject matter is the use of an interactive individualized device for judging the effectiveness of various interventions for hyperlipidemia.

Hyperlipidemia is known to be a major risk factor in the development of cardiovascular diseases. It is possible for the physician to therefore calculate a threshold target for each of the lipoproteins affecting hyperlipidemia based on prescribing different treatment options, such as lifestyle modification, exercise, or various medications for patients.

A patient or a physician can input the patient's own laboratory values for serum lipoprotein measurements at the point of care to evaluate the predicted response to various treatment interventions. Based upon standard lipid treatment goals for this patient as recommended by the National Cholesterol Education Project Adult Treatment Panel III, it was determined that his treatment targeted goals for TOTAL CHOLESTEROL were 199 or less, TRIGLYCERIDES 150 or less, LDL CHOLESTEROL 99 or less, and HDL CHOLESTEROL of 50 greater. Table 1 lists the patient's baseline laboratory test results for limited lipoprotein analysis, but the device could have easily entered an entire lipoprotein profile for treatment analysis.

TABLE 1
PATIENT BASELINE LIPOPROTEIN LEVELS

| TOTAL | TRIGLYC- | HDL | LDL |
|---|---|---|---|
| CHOLESTEROL | ERIDES | CHOLESTEROL | CHOLESTEROL |
| 283 | 200 | 44 | 199 |

Entering the above patient data in the patient data entry field of the calculator generates a “new” set of post treatment predictive values for “Total Cholesterol”, “Triglycerides”, “LDL Cholesterol”, and “HDL cholesterol”. These are displayed in the right hand columns below in table 2. It is readily and very quickly apparent that the interventions “AHA Step I diet”, “AHA Step II diet”, “Pritiken diet”, “Aerobic Exercise”, “Fluvastatin 80”, “Lovastatin 20 or 40”, “Pravastatin 20, 40, 80”, “Simvastatin 10, 20 or 40”, “Atorvastatin 10 or 40”, “Cholestyramine”, “Gemfibrizol”, “Fenofibrate 145”, “Ezetemibe 10”, “Colesevelam 3.8 GM”, “Niaspan® at any dose”, “Niaspan 2GM+statin”, and “Niaspan 2 GM+Bile Acid Sequestrant” will not bring the four parameters into the targeted range. However, “Simvastatin 80”, “Atorvastatin 80”, and “Rosuvastatin 20 or 40” normalize three of the four parameters into the target range. “Vytorin® 10/20, 10/40, and 10/80” normalizes each parameter into the normal range except for the HDL Cholesterol.

The post treatment values (“new LDL Cholesterol” etc) are displayed in the four right most columns in Table 2. It is readily determined by glancing down each column and comparing the target treatment goals with the predictions that this patient can achieve the desired target treatment goals with several interventions, but that the likelihood of success with other interventions would not be as desirable. Having the columns in different colors facilitates the user in quickly researching the results. The patient was able to select from a number of possible interventions.

In Table 2, it can be appreciated that “Aerobic Exercise” raises the HDL to 52.36 which is in the target range. Alternatively, any dose of “Niacin” or “Niaspan®” will elevate the HDL into the targeted range. Therefore, it becomes easy for this patient to assess that a combination of “Aerobic Exercise” and “Vytorin-any dose” would be quite likely successful in achieving the target ranges for all four parameters. Alternatively, “Niacin” or “Niaspan®” plus any dose of “Vytorin®” would likely result in successful manipulation of these variables. The patient would then hyperlink to the pages for “Vytorin®”, “Aerobic Exercise”, “Niaspan®”, and “Niacin” to read a description of the treatment along with side effects of each. There he would learn that “Aerobic Exercise” required to achieve the desired HDL goal would be defined as minimum 30 minute exercise sessions of walking jogging, bicycling, stair climbing, rowing, or swimming in which the heart rate reaches and maintains 70% of the maximum predicted for age, at least 4 days per week. He would also learn that “Vytorin” in any dose carries, among others, possible side effects including muscle aching, liver function test abnormalities, and rhabdomyolysis and that the higher the dose, the more likely the side effect. He would also learn that combining “Vytorin®” with “Niaspan®” or “Niacin” increases the likelihood of these events. Lastly, he would be able to determine the cost of “Vytorin®” per month through the pharmacy benefits package of his particular health insurer. Given this information, he may well decide that a program of “Aerobic Exercise” and “Vytorin® 10/10”, “Vytorin® 10/20” or “Vytorin® 10/40” would be best for him.

However, it is conceivable that “Vytorin®” may not be on the formulary of the patient's insurance plan. Then a copy of the treatment plan can be printed and mailed, E-mailed, or sent via other secure digital means to the insurer along with a request for “non-formulary coverage” pre-approval on the grounds that this is the only agent likely to achieve success for this patient without having to go through the usual trial and error approach of using each agent that is on the formulary prior to finally trying the non-formulary agent, which is in this case the only one likely to be successful.

Another possibility for this particular patient would be to commit to a treatment regimen of both “Aerobic Exercise” and “AHA Step I diet”. The regime assumes adherence to this dietary and exercise plan by the patient and predicts a follow-up treatment “failure” as defined by inability to achieve targeted values for lipoproteins at the follow-up appointment. Assuming adherence to this therapy, the baseline of liproprotein for this patient in Table 1 will result in lipoprotein follow-up levels at 6 weeks of Total Cholesterol 254, Triglyceride 184, LDL Cholesterol 175, and HDL cholesterol 52 (the effect of “Aerobic Exercise”).

The patient might then input these values into the treatment calculator as a new “Baseline Value” during the first physician visit as depicted in Table 3. Utilizing the treatment tool in Table 3, it is now possible to predict that the addition of many more medication interventions has a high probability of success. It can be seen from Table 3 that with the addition of the “lifestyle modification treatments” of both “AHA Step I diet” and “Aerobic Exercise”, the addition of “Simvistatin 80”, “Atorvastatin 20, 40 or 80”, “Rosuvastatin 10 or 40”, or “Vytorin® 10/10, 10/20, 10/40, or 10/80” now will result in normalization of all lipoprotein parameters. This allows the patient more choices on his current insurance plan. The patient can reduce his out-of-pocket costs by paying a lower co-payment for his medications by adopting the lifestyle modifications of “Aerobic Exercise” and “AHA Step I diet”. Once the option is chosen, the appropriate prescriptions and patient education material can be communicated wirelessly to an external source, such as a central medical server, a printer, or a PDA.

TABLE 2
EFFECT OF LIPID LOWERING REGIMENS-PREDICTED VALUES
BASED UPON INTERVENTION

| | PATIENT LIPID VALUES (BASELINE) | | | |
|---|---|---|---|---|
| | Type in patient | | | |
| | baseline value here | | | |
| | | | LDL | HDL |
| | TCHOL | TRIG | CHOL | CHOL |
| | 283 | 200 | 199 | 44 |
| INTERVENTION | NEW TC | NEW TG | NEW | NEW |
| | | | LDL | HDL |
| AHA STEP I DIET | 254.7 | 184 | 175.12 | 44 |
| AHA STEP II DIET | 246.21 | 182 | 171.14 | 44 |
| PRITIKEN DIET | 228.381 | 118 | 153.23 | 37.84 |
| AEROBIC | | | | 52.36 |
| EXERCISE | | | | |
| FLUVASTATIN 80 | 212.25 | 150 | 133.33 | 48.84 |
| LOVASTATIN 20 | 229.23 | 182 | 145.27 | 46.64 |
| LOVASTATIN 40 | 220.74 | 184 | 137.31 | 46.2 |
| PRAVASTATIN 20 | 215.08 | 178 | 135.32 | 44.88 |
| PRAVASTATIN 40 | 212.25 | 152 | 131.34 | 49.28 |
| PRAVASTATIN 80 | | | | |
| SIMVISTATIN 5 | 229.23 | 176 | 147.26 | 48.4 |
| SIMVISTATIN 10 | 217.91 | 170 | 139.3 | 49.28 |
| SIMVISTATIN 20 | 203.76 | 162 | 123.38 | 47.52 |
| SIMVISTATIN 40 | 195.27 | 164 | 117.41 | 49.72 |
| SIMVISTATIN 80 | 181.12 | 152 | 105.47 | 51.04 |
| ATORVASTATIN 10 | 203.76 | 118 | 123.38 | 50.16 |
| ATORVASTATIN 20 | 186.78 | 122 | 107.46 | 48.84 |
| ATORVASTATIN 40 | 217.91 | 142 | 99.5 | 45.76 |
| ATORVASTATIN 80 | 158.48 | 96 | 77.61 | 43.56 |
| ROSUVASTATIN 10 | 169.8 | 126 | 109.45 | 47.52 |
| ROSUVASTATIN 20 | 186.78 | 126 | 137.31 | 53.68 |
| ROSUVASTATIN 40 | 169.8 | 114 | 113.43 | 51.48 |
| CHOLESTYRAMINE | 234.89 | 222 | 153.23 | 47.52 |
| GEMFIBROZIL 600 | 271.68 | 138 | 238.8 | 51.48 |
| BID | | | | |
| FENOFIBRATE 145 MG | 229.23 | 142 | 159.2 | 50.6 |
| EZETIMIBE 10 MG | 249.04 | 182 | 163.18 | 44.44 |
| COLESEVELAM 3.8 GM | 263.19 | 180 | 169.15 | 45.32 |
| NIASPAN ® 500 MG | 277.34 | 190 | 193.03 | 48.4 |
| DAILY | | | | |
| NIASPAN ® 750 MG | | | | |
| DAILY | | | | |
| NIASPAN ® 1000 MG | 268.85 | 178 | 181.09 | 50.16 |
| DAILY | | | | |
| NIASPAN ® 1500 MG | 251.87 | 144 | 171.14 | 53.68 |
| DAILY | | | | |
| NIASPAN ® 2000 MG | 249.04 | 130 | 165.17 | 58.08 |
| DAILY | | | | |
| NIACIN 500 MG | 254.7 | 160 | 179.1 | 50.6 |
| NIACIN 1 GM | 240.55 | 140 | 169.15 | 52.8 |
| NIACIN 1.5 GM | 226.4 | 120 | 159.2 | 57.2 |
| NIACIN 2 GM | 226.4 | 100 | 159.2 | 59.4 |
| COMBINATIONS | | | | |
| NIASPAN ® | 240.55 | 210 | 163.18 | 57.64 |
| 2G + STATIN | | | | |
| VYTORIN ® 10/80 | 166.97 | 138 | 83.58 | 49.28 |
| VYTORIN ® 10/20 | 181.12 | 144 | 99.5 | 47.96 |
| VYTORIN ® 10/40 | 172.63 | 136 | 87.56 | 48.84 |

TABLE 3
EFFECT OF LIPID LOWERING REGIMENS-PREDICTED VALUES BASED UPON
INTERVENTION

| Type in patient | PATIENT LIPID VALUES (BASELINE) | | | |
|---|---|---|---|---|
| baseline value here --- | TCHOL | TRIG | LDL CHOL | HDL CHOL |
| ---> | 254.7 | 184 | 175 | 52.36 |
| INTERVENTION | NEW TC | NEW TG | NEW LDL | NEW HDL |
| AHA STEP I DIET | 229.23 | 169.28 | 154 | 52.36 |
| AHA STEP II DIET | 221.589 | 167.44 | 150.5 | 52.36 |
| PRITIKEN DIET | 205.5429 | 108.56 | 134.75 | 45.0296 |
| AEROBIC | | | | 62.3084 |
| EXERCISE | | | | |
| FLUVASTATIN 80 | 191.025 | 138 | 117.25 | 58.1196 |
| LOVASTATIN 20 | 206.307 | 167.44 | 127.75 | 55.5016 |
| LOVASTATIN 40 | 198.666 | 169.28 | 120.75 | 54.978 |
| PRAVASTATIN 20 | 193.572 | 163.76 | 119 | 53.4072 |
| PRAVASTATIN 40 | 191.025 | 139.84 | 115.5 | 58.6432 |
| PRAVASTATIN 80 | | | | |
| SIMVASTATIN 5 | 206.307 | 161.92 | 129.5 | 57.596 |
| SIMVASTATIN 10 | 196.119 | 156.4 | 122.5 | 58.6432 |
| SIMVASTATIN 20 | 183.384 | 149.04 | 108.5 | 56.5488 |
| SIMVASTATIN 40 | 175.743 | 150.88 | 103.25 | 59.1668 |
| SIMVASTATIN 80 | 163.008 | 139.84 | 92.75 | 60.7376 |
| ATORVASTATIN 10 | 183.384 | 108.56 | 108.5 | 59.6904 |
| ATORVASTATIN 20 | 168.102 | 112.24 | 94.5 | 58.1196 |
| ATORVASTATIN 40 | 196.119 | 130.64 | 87.5 | 54.4544 |
| ATORVASTATIN 80 | 142.632 | 88.32 | 68.25 | 51.8364 |
| ROSUVASTATIN 10 | 152.82 | 115.92 | 96.25 | 56.5488 |
| ROSUVASTATIN 20 | 168.102 | 115.92 | 120.75 | 63.8792 |
| ROSUVASTATIN 40 | 152.82 | 104.88 | 99.75 | 61.2612 |
| CHOLESTYRAMINE | 211.401 | 204.24 | 134.75 | 56.5488 |
| GEMFIBRIZ 600 | 244.512 | 126.96 | 210 | 61.2612 |
| FENOFIBRATE 145 MG | 206.307 | 130.64 | 140 | 60.214 |
| EZETIMIBE 10 MG | 224.136 | 167.44 | 143.5 | 52.8836 |
| COLESEVELAM 3.8 GM | 236.871 | 165.6 | 148.75 | 53.9308 |
| NIASPAN ® 500 MG | 249.606 | 174.8 | 169.75 | 57.596 |
| DAILY | | | | |
| NIASPAN ® 750 MG | | | | |
| DAILY | | | | |
| NIASPAN ® 1000 MG | 241.965 | 163.76 | 159.25 | 59.6904 |
| DAILY | | | | |
| NIASPAN ® 1500 MG | 226.683 | 132.48 | 150.5 | 63.8792 |
| DAILY | | | | |
| NIASPAN ® 2000 MG | 224.136 | 119.6 | 145.25 | 69.1152 |
| DAILY | | | | |
| NIACIN 500 MG | 229.23 | 147.2 | 157.5 | 60.214 |
| NIACIN 1 GM | 216.495 | 128.8 | 148.75 | 62.832 |
| NIACIN 1.5 GM | 203.76 | 110.4 | 140 | 68.068 |
| NIACIN 2 GM | 203.76 | 92 | 140 | 70.686 |
| COMBINATIONS | | | | |
| NIASPAN ® 2 G + BAS | 216.495 | 193.2 | 143.5 | 68.5916 |
| NIASPAN ® | 193.572 | 125.12 | 119 | 65.45 |
| 2 G + STATIN | | | | |
| VYTORIN ® 10/10 | 175.743 | 141.68 | 96.25 | 56.5488 |
| VYTORIN ® 10/80 | 150.273 | 126.96 | 73.5 | 58.6432 |
| VYTORIN ® 10/20 | 163.008 | 132.48 | 87.5 | 57.0724 |
| VYTORIN ® 10/40 | 155.367 | 125.12 | 77 | 58.1196 |

PREDICTED RESPONSE OF LIPOPROTEINS TO VARIOUS THERAPEUTIC OPTIONS

| | | | | | | | VALUES | |
|---|---|---|---|---|---|---|---|---|
| | T | | LDL | HDL | PATIENT | LIPID | LDL | (BASELINE) |
| | CHOL | TRIGLY | CHOL | CHOL | T CHOL | TRIG | CHOL | HDL CHOL |
| | 229 | 63 | 134 | 65 | 229 | 63 | 134 | 65 |
| INTERVENTION | TOT | TRIGLY | LDL- | HDL | NEW TC | NEW | NEW | NEW HDL |
| | CHOL | | CHOL | CHOL | | TG | LDL | |
| AHA STEP I DIET | −10% | −8% | −12% | 0% | 206.1 | 57.96 | 117.92 | 65 |
| AHA STEP II DIET | −13% | −9% | −16% | 0% | 199.23 | 57.33 | 115.24 | 65 |
| PRITIKEN DIET | −19.3 | −41% | −23% | −14% | 184.803 | 37.17 | 103.18 | 55.9 |
| AEROBIC EXERCISE | | | | 19% | | | | 77.35 |
| FLUVASTATIN 20 MG | −17% | −12% | −22% | 3% | 190.07 | 55.44 | 104.52 | 66.95 |
| FLUVASTATIN 40 MG | −19% | −14% | −25% | 4% | 185.49 | 54.18 | 100.5 | 67.6 |
| FLUVASTATIN 80 MG | −25% | −19% | −35% | 7% | 171.75 | 51.03 | 87.1 | 69.55 |
| LOVASTATIN 10 MG | −16% | −10% | −21% | 5% | 192.36 | 56.7 | 105.86 | 68.25 |
| LOVASTATIN 20 MG | −19% | 9% | −27% | 6% | 185.49 | 57.33 | 97.82 | 68.9 |
| LOVASTATIN 40 MG | −22% | −8% | −31% | 5% | 178.62 | 57.96 | 92.46 | 68.25 |
| PRAVASTATIN 10 MG | −16% | −15% | −22% | 7% | 192.36 | 53.55 | 104.52 | 69.55 |
| PRAVASTATIN 20 MG | −24% | −11% | −32% | 2% | 174.04 | 56.07 | 91.12 | 66.3 |
| PRAVASTATIN 40 MG | −25% | −24% | −34% | 12% | 171.75 | 47.88 | 88.44 | 72.8 |
| PRAVASTATIN 80 MG | | | | | | | | |
| SIMVISTATIN 5 MG | −19% | −12% | −26% | 10% | 185.49 | 55.44 | 99.16 | 71.5 |
| SIMVISTATIN 10 MG | −23% | −15% | −30% | 12% | 176.33 | 53.55 | 93.8 | 72.8 |
| SIMVISTATIN 20 MG | −28% | −19% | −38% | 8% | 164.88 | 51.03 | 83.08 | 70.2 |
| SIMVISTATIN 40 MG | −31% | −18% | −41% | 9% | 158.01 | 51.66 | 79.06 | 70.85 |
| SIMVISTATVN 80 MG | −36% | −24% | −47% | 8% | 146.56 | 47.88 | 71.02 | 70.2 |
| ATORVASTATIN 10 MG | −29% | −41% | −38% | 14% | 162.59 | 37.17 | 83.08 | 74.1 |
| ATORVASTATIN 20 MG | −33% | −26% | −43% | 9% | 153.43 | 46.62 | 76.38 | 70.85 |
| ATORVASTATIN 40 MG | −37% | −29% | −50% | 6% | 144.27 | 44.73 | 67 | 68.9 |
| ATORVASTATIN 80 MG | −45% | −37% | −60% | 5% | 125.95 | 39.69 | 53.6 | 68.25 |
| ROSUVASTATIN 5 MG | −33% | −35% | −45% | 13% | 153.43 | 40.95 | 73.7 | 73.45 |
| ROSUVASTATIN 10 MG | −36% | −52% | −45% | 14% | 146.56 | 30.24 | 73.7 | 74.1 |
| ROSUVASTATIN 20 MG | −40% | −23% | −55% | 8% | 137.4 | 48.51 | 60.3 | 70.2 |
| ROSUVASTATIN 40 MG | −46% | −28% | −63% | 10% | 123.66 | 45.36 | 49.58 | 71.5 |
| CHOLESTYRAMINE | −17% | 11% | −23% | 8% | 190.07 | 69.93 | 103.18 | 70.2 |
| GEMFIBRIZOL 600 | −4% | −31% | 20% | 17% | 219.84 | 43.47 | 160.8 | 76.05 |
| BID | | | | | | | | |
| FENOFIBRATE 145 MG | −19% | −29% | −20% | 11% | 185.49 | 44.73 | 107.2 | 72.15 |
| EZETIMIBE 10 MG | −13% | −11% | −19% | 5% | 199.23 | 56.07 | 108.54 | 68.25 |
| COLESEVELAM 3.8 GM | −7% | 10% | −15% | 3% | 212.97 | 56.7 | 113.9 | 66.95 |
| COLESEVELAM 4.5 GM | −10% | 9% | −18% | 3% | 206.1 | 68.67 | 109.88 | 66.95 |
| NIASPAN ® 500 MG | −2% | −5% | −3% | 10% | 224.42 | 59.85 | 129.98 | 71.5 |
| DAILY | | | | | | | | |
| NIASPAN ® 1000 MG | −5% | −11% | −9% | 15% | 217.55 | 56.07 | 121.94 | 74.75 |
| DAILY | | | | | | | | |
| NIASPAN ® 1500 MG | −11% | −28% | −14% | 22% | 203.81 | 45.36 | 115.24 | 79.3 |
| DAILY | | | | | | | | |
| NIASPAN ® 2000 MG | −12% | −35% | −17% | 26% | 201.52 | 40.95 | 111.22 | 81.9 |
| DAILY | | | | | | | | |
| NIACIN 500 MG | −10% | −20% | −10% | 15% | 206.1 | 50.4 | 120.6 | 74.75 |
| NIACIN 1 GM | −15% | −30% | −15% | 20% | 194.65 | 44.1 | 113.9 | 78 |
| NIACIN 1.5 GM | −20% | −40% | −20% | 30% | 183.2 | 37.8 | 107.2 | 84.5 |
| NIACIN 2 GM | −20% | −50% | −20% | 35% | 183.2 | 31.5 | 107.2 | 87.75 |
| COMBINATIONS | | | | | | | | |
| NIASPAN ® 2 G + BAS | −15% | 5% | −28% | 31% | 194.65 | 66.15 | 109.88 | 85.15 |
| NIASPAN ® | −24% | −32% | −32% | 25% | 174.04 | 42.84 | 91.12 | 81.25 |
| 2 G + STATIN (ANY) | | | | | | | | |
| NIASPAN ® | | −32% | −30% | 20% | | 42.84 | 93.8 | 78 |
| 1 G/LOVASTATIN 20 | | | | | | | | |
| NIASPAN ® | | −39% | −36% | 20% | | 38.43 | 85.76 | 78 |
| 1 G/LOVASTATIN 40 | | | | | | | | |
| NIASPAN ® | | −44% | −37% | 27% | | 35.28 | 84.42 | 82.55 |
| 1.5 G/LOVASTATIN 40 | | | | | | | | |
| NIASPAN ® 2 G/ | | −44% | −42% | 30% | | 35.28 | 77.72 | 84.5 |
| LOVASTATIN 40 | | | | | | | | |
| VYTORIN ® 10/10 | −31% | −23% | −45% | 8% | 158.01 | 48.51 | 73.7 | 70.2 |
| VYTORIN ® 10/20 | −36% | −28% | −50% | 9% | 146.56 | 45.36 | 67 | 70.85 |
| VYTORIN ® 10/40 | −39% | −32% | −56% | 11% | 139.69 | 42.84 | 58.96 | 72.15 |
| VYTORIN ® 10/80 | −43% | −31% | −58% | 12% | 130.53 | 43.47 | 56.28 | 72.8 |

### Example 2

### Patient with Hypertension or High Blood Pressure

There are currently defined four classifications of blood pressure. The first classification is normal blood pressure, defined as systolic blood pressure of less than 120 and diastolic blood pressure of less than 80. The next classification is “Prehypertension” and is defined as a systolic blood pressure of between 120 and 139 and a diastolic blood pressure of between 80 and 89 mmHg. The third classification, “Stage I hypertension” is defined as a systolic blood pressure of between 140 and 159 and a diastolic blood pressure of between 90 and 99. The final classification, “Stage II hypertension” is defined as a systolic blood pressure of greater than or equal to 160 and a diastolic blood pressure of greater than or equal to 100.

Various treatments are available to reduce the stage of hypertension from Stage II to Stage I to Prehypertension and finally to normal. Some of these involve lifestyle modification, such as weight reduction, dietary sodium restriction, the DASH® diet, physical activity, and moderation of alcohol consumption. There are also currently around sixty different blood pressure medications available to the patient and practicing physician, half of which are available generically (and therefore at lower cost). Many of the available medications are combined in various proprietary preparations as well, leaving a vast array of treatment options available for the clinician. Certain testing, follow-up monitoring, and special circumstances are recommended to monitor specific blood pressure.

The treatment planning device, a portion of which is depicted in Table 4, allows the patient and physician to collaborate on the selection of a medical regime. To a viewer, it is readily apparent that the particular patient with Stage I hypertension and a compelling indication, such as diabetes or left ventricular hypertrophy (LVH), for a specific drug regimen, would benefit from lifestyle modification in the form of physical activity and sodium restriction but would be unlikely to achieve the target blood pressure of less than 120 systolic without significant weight loss. The treatment planning device would allow the patient and physician to set specific goals of aerobic exercise, sodium restriction to less than 3000 mg dietary sodium per day and weight loss of 5 Kg (11 pounds) by the next office visit, anticipating a net total maximum drop in systolic blood pressure in that interval of 27 mmHg and minimum drop in systolic blood pressure in that interval of 9 mmHg. If the maximum is achieved, the patient would reach the treatment goal blood pressure, with a final target BP of 115/87. If only the minimum is achieved, the patient would further the use of the device to set a new goal as exhibited in Table 5.

Assuming the patient complies with the treatment plan, by partaking the sodium restriction, weight loss, and exercise portions for eight weeks, but only achieved minimum changes in blood pressure predicted (systolic blood pressure reading of 133.5). The “new” baseline blood pressure readings can then be entered into the treatment calculator at the next visit, as depicted in Table 5. Upon entering this new data, it can be readily appreciated that the device predicts that the addition of Felodipine at 10 mg daily, Isradapine 15 or 20 mg daily, or Nisoldipine 60 mg daily will allow LIS to approximately reach the goal set if maximum efficacy is achieved. It will be understood by those skilled in the art that Table 4 represents only a portion of the therapeutic choices available to this patient and that the actual treatment planning device would list and apply these variables as well to the particular patient involved here. This might result in some additional therapeutic choices for this patient. In the abbreviated device visualized in Table 4 and Table 5, however, these are the choices from which a patient and physician could select. To proceed with this example, and in a manner similar to that explained in Example 1 above, the patient would then hyperlink to a page examining various side effects of these medications. The patient or the patient's pharmacy is provided with a treatment plan, cost plan, and follow-up plans. These plans can be communicated to an external source wirelessly, even to a central medical server or an insurance provider.

TABLE 4
EFFECT OF ANTIHYPERTENSIVE INTERVENTION BASED UPON BASELINE
PRETREATMENT BP

| Type in | | | | | | |
|---|---|---|---|---|---|---|
| Baseline Here | SYSTOLIC | DIASTOLIC | NEW | NEW | NEW | NEW |
| -----------> | 142 | 96 | WORSE | WORST | BEST CASE | BEST CASE |
| | | | CASE | CASE | SYSTOLIC | DIASTOLIC |
| | | | SYSTOLIC | DIASTOLIC | | |
| Interventions | | | | | | |
| WEIGHT | | | | | | |
| REDUCTION- | | | | | | |
| IDEAL | | | | | | |
| BMI 18.5-24.9 KG/M | | | | | | |
| SQ. | | | | | | |
| WEIGHT LOSS | | | | | | |
| GOAL IN KG | | | | | | |
| ENTER HERE | | | | | | |
| 5 KG WT | | | 139.5 | 96 | 132 | 89 |
| LOSS | | | | | | |
| PRESCRIBED | | | | | | |
| DIETARY | | | 140 | 96 | 134 | 89 |
| SODIUM | | | | | | |
| RESTRICTION | | | | | | |
| DASH DIET | | | 134 | 96 | 128 | 89 |
| AEROBIC | | | 138 | 96 | 133 | 89 |
| EXERCISE | | | | | | |
| MODERATION | | | 140 | 96 | 138 | 89 |
| OF ALCOHOL | | | | | | |
| COMBINATION | | | | | | |
| WT LOSS 5 KG | | | | | | |
| SODIUM | | | 133.5 | 96 | 115 | 89 |
| RESTRICTION | | | | | | |
| AND | | | | | | |
| EXERCISE | | | | | | |
| CALCIUM | | | | | | |
| CHANNEL | | | | | | |
| BLOCKER | | | | | | |
| AMLODIPINE | | | | | | |
| (NORVASC ®) | | | | | | |
| 5 MG | | | 121.5 | 83 | 120.5 | 82 |
| 10 MG | | | 121.5 | 83 | 120.5 | 82 |
| FELODIPINE | | | | | | |
| (PLENDIL ®) | | | | | | |
| 2.5 MG | | | 128.8 | 86.5 | 124.1 | 86.3 |
| 5 MG | | | 127.2 | 86.6 | 124 | 85.3 |
| 10 MG | | | 122.7 | 80.4 | 115.5 | 79 |
| ISRADAPINE | | | | | | |
| (DYNACIRCCR ®) | | | | | | |
| 5 MG | | | 128.3 | 86.2 | 128.3 | 86.2 |
| 10 MG | | | 120.1 | 79.3 | 120.1 | 79.3 |
| 15 MG | | | 117.9 | 78.8 | 117.9 | 78.8 |
| 20 MG | | | 118 | 77.2 | 118 | 77.2 |
| NIFEDIPINE | | | | | | |
| LA (ADALATCC ®) | | | | | | |
| 30 MG | | | 128.2 | 86.1 | 128.2 | 86.1 |
| 60 MG | | | 125.5 | 84.9 | 125.5 | 84.9 |
| 90 MG | | | 121 | 80.9 | 121 | 80.9 |
| NISOLDIPINE | | | | | | |
| (SULAR ®) | | | | | | |
| 10 MG | | | 125.5 | 86 | 125.5 | 86 |
| 20 MG | | | 122.5 | 84 | 122.5 | 84 |
| 30 MG | | | 122.5 | 82 | 122.5 | 82 |
| 40 MG | | | 119.5 | 82 | 119.5 | 82 |
| 60 MG | | | 118.5 | 79 | 118.5 | 79 |

TABLE 5
EFFECT OF ANTIHYPERTENSIVE INTERVENTION BASED UPON BASELINE
PRETREATMENT BP

| Type in | | | | | | |
|---|---|---|---|---|---|---|
| Baseline | | | | | | |
| Here | SYSTOLIC | DIASTOLIC | NEW | NEW | NEW | NEW |
| -----------> | 133.5 | 89 | WORSE | WORST | BEST CASE | BEST CASE |
| | | | CASE | CASE | SYSTOLIC | DIASTOLIC |
| | | | SYSTOLIC | DIASTOLIC | | |
| Interventions | | | | | | |
| CALCIUM | | | | | | |
| CHANNEL | | | | | | |
| BLOCKER | | | | | | |
| AMLODIPINE | | | | | | |
| (NORVASC ®) | | | | | | |
| 5 MG | | | 121.5 | 83 | 120.5 | 82 |
| 10 MG | | | 121.5 | 83 | 120.5 | 82 |
| FELODIPINE | | | | | | |
| (PLENDIL ®) | | | | | | |
| 2.5 MG | | | 128.8 | 86.5 | 124.1 | 86.3 |
| 5 MG | | | 127.2 | 86.6 | 124 | 85.3 |
| 10 MG | | | 122.7 | 80.4 | 115.5 | 79 |
| ISRADAPINE | | | | | | |
| (DYNACIRCCR ®) | | | | | | |
| 5 MG | | | 128.3 | 86.2 | 128.3 | 86.2 |
| 10 MG | | | 120.1 | 79.3 | 120.1 | 79.3 |
| 15 MG | | | 117.9 | 78.8 | 117.9 | 78.8 |
| 20 MG | | | 118 | 77.2 | 118 | 77.2 |
| NIFEDIPINE | | | | | | |
| LA (ADALATCC ®) | | | | | | |
| 30 MG | | | 128.2 | 86.1 | 128.2 | 86.1 |
| 60 MG | | | 125.5 | 84.9 | 125.5 | 84.9 |
| 90 MG | | | 121 | 80.9 | 121 | 80.9 |
| NISOLDIPINE | | | | | | |
| (SULAR ®) | | | | | | |
| 10 MG | | | 125.5 | 86 | 125.5 | 86 |
| 20 MG | | | 122.5 | 84 | 122.5 | 84 |
| 30 MG | | | 122.5 | 82 | 122.5 | 82 |
| 40 MG | | | 119.5 | 82 | 119.5 | 82 |
| 60 MG | | | 118.5 | 79 | 118.5 | 79 |

Thus, specific embodiments and applications of the device have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A device comprising:
a first interface that receives patient-specific information;
a second interface that displays a plurality of treatment options; and
a processor that executes software that projects clinical outcomes for at least first and second ones of the treatment options as a function of historic data and the patient-specific information; and
a third interface that displays at least the first and second ones of the treatment options.

2. The device of claim 1, wherein at least one of the first and second interfaces comprises an electronic display

3. The device of claim 1, wherein the third interface comprises a printout.

4. The device of claim 1, wherein the second and third interfaces comprise a spreadsheet.

5. The device of claim 1, wherein the first and second outcomes comprise at least one of a change in severity, frequency, or duration of a symptom.

6. The device of claim 1, wherein the first and second outcomes comprise at least one of a change in a laboratory value.

7. The device of claim 1, wherein the processor further executes the software to project a monetary cost associated with at least one of the first and second ones of the treatment options.

8. The device of claim 1, wherein the processor further executes the software to identify a side effect associated with at least one of the first and second ones of the treatment options.

9. The device of claim 1, wherein the processor further executes the software to identify likelihood of occurrence of the side effect.

10. The device of claim 1, further comprising a fourth interface that receives a diagnosis.

11. The device of claim 10, wherein the processor further executes the software to project the clinical outcomes as a function of the diagnosis.

11. The device of claim 1, further comprising a fourth interface that displays a plurality of diagnostic possibilities.

12. The device of claim 11, further comprising a fourth interface that correlates costs with the diagnostic possibilities.

13. The device of claim 11, further comprising a fourth interface that correlates at least the first and second ones of the treatment options with the diagnostic possibilities.

14. The device of claim 1, wherein at least one of the first and second treatment options comprises administration of a drug.

15. The device of claim 1, wherein at least one of the first and second treatment options comprises a lifestyle change.

16. The device of claim 1, comprising a pocket sized housing.

17. The device of claim 1, comprising a display screen.

18. The device of claim 1, further comprising a microprocessor.

19. The device of claim 1, further comprising a memory.

20. The device of claim 1, further comprising control buttons.

21. A method of advising a patient, comprising:
entering into the device clinical information specific to the patient;
retrieving from the device a comparison of treatment options and clinical outcomes that have historically been associated with change of a value within the clinical information; and
relating at least a portion of the comparison to the patient.

22. The method of claim 21, wherein the clinical outcomes include at least one of a change in severity, frequency, or duration of a symptom.

23. The method of claim 21, wherein the value comprises a laboratory test result.

24. The method of claim 21, wherein the clinical outcomes include a monetary cost.

25. The method of claim 21, wherein the step of retrieving a comparison from the device comprising a user of the device viewing a display that displays at least a portion of the comparison.

26. The method of claim 21, wherein the step of retrieving a comparison from the device comprising printing at least a portion of the comparison.

27. The method of claim 21, further comprising entering into the device a diagnosis that is specific to the patient.

28. The method of claim 21, further comprising a medical professional using the comparison as a basis from which to discuss at least some of the treatment options and at least some of the clinical outcomes with the patient.

29. The method of claim 21, wherein the device has sufficient processing power and memory such that the device does not need to communicate with an external source to produce the comparison.

30. The method of claim 21, further comprising an interface through which the device communicates to an external source to produce the comparison.
